(19) United States
(12) Patent Application Publication
(10) Pub. No.: US 2010/0119561 A1
(43) Pub. Date: May 13, 2010

(54) Compositions Having a Performance Indicator

(75) Inventors: Ralph Spindler, Palatine, IL (US); Nataliya V. Larionova, Evanston, IL (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: AMCOL International Corporation, Hoffman Estates, IL (US)

(21) Appl. No.: 12/443,998

(22) PCT Filed: Oct. 4, 2007
(86) PCT No.: PCT/US07/80403

§ 371 Date: Jan. 5, 2010

Related U.S. Application Data
(60) Provisional application No. 60/849,985, filed on Oct. 5, 2006.

(51) Int. Cl. A61K 8/02; A61K 9/14; A61K 31/164; A61K 31/60; A61K 38/43; A61K 38/16; A61K 31/573; A61P 1/02; C08K 3/34; A61K 9/50; C09K 11/02; C11D 3/40

(52) U.S. Cl. 424/401; 424/489; 514/626; 514/159; 424/94.1; 514/12; 514/179; 524/443; 424/490; 252/301.36; 510/100; 424/49

(57) ABSTRACT

Compositions containing a delivery system including polymeric microparticles capable of undergoing a visual change, such as a color change, after application to a substrate are disclosed. The delivery system informs a consumer that the composition has been properly applied and/or has performed its intended function.

### CROSS-REFERENCE TO RELATED APPLICATION

This application claims the benefit of U.S. provisional patent application No. 60/849,985, filed Oct. 5, 2006, incorporated herein by reference.

### FIELD OF THE INVENTION

The present invention relates to personal care, oral care, toiletries, topical cleaning, and pharmaceutical compositions containing a visual indicator. During use, a consumer can observe a color change after application of the composition, which indicates that the composition has been properly applied and/or that the composition has performed its intended function.

### BACKGROUND OF THE INVENTION

Providing a composition capable of informing a consumer that the composition has been properly applied and/or applied for a sufficient period of time to provide a benefit is a difficult challenge. Often a consumer applies a composition, for example, a hand wash to the skin, for a short period of time, whereas a longer contact period would provide a more efficacious removal of bacteria and soil, e.g., a more complete cleaning. This is especially important in oral care products, like toothpastes, wherein a minimum two minute brushing is recommended, but often the actual brushing time is significantly less. Therefore, a need exists to provide a visual cue that application of the composition is thorough, and remains applied for a sufficiently time to achieve its intended benefit.

Several approaches for introducing visual indicators into oral care products have been attempted. In U.S. Pat. No. 4,150,106, a change in pH is used to generate a color change in a pH-sensitive dye, such that upon application of a toothpaste, the color changes from yellow to purple as the acidic toothpaste interacts with saliva to increase pH. However, the pH change required for this type of product can limit formulation flexibility and/or compatibility of ingredients that may not be stable under the low pH conditions required for the toothpaste.

U.S. Pat. No. 6,419,902 discloses a different approach, wherein individual stripes of different dyes or pigments are present in a single toothpaste formulation. When the consumer applies the toothpaste, the two colors mix to produce a third color as an indicator that brushing is complete. This approach requires that the individual colors do not bleed into one another; otherwise the toothpaste will change to the indicator color prematurely. Adding stripes into a toothpaste product also can increase the cost of the toothpaste, both from the standpoint of producing different compositions that form the stripes included in a single tube, and the need to use expensive processing equipment to manufacture the final composition.

Delivery systems often are used in personal care and pharmaceutical topical compositions to extend release of an active ingredient, to protect an active ingredient from degradation in the composition, and/or to enable formulation of the active ingredient into the composition due to difficulties, such as solubility or formulation esthetics. One type of delivery system that can achieve these goals in a composition is the polymeric microparticle delivery systems. One class of adsorbent polymeric microparticles is prepared by suspension polymerization techniques, as set forth in U.S. Pat. Nos. 5,677,407; 5,712,358; 5,777,054; 5,830,967; 5,834,577; 5,955,552; and 6,107,429, each incorporated herein by reference (available commercially under the tradename of POLY-PORE® E200, NCI name, ally methacrylates crosspolymer, from AMCOL International, Arlington Heights, Ill.). Another class of adsorbent polymeric microparticles is prepared by a precipitation polymerization technique, as set forth in U.S. Pat. Nos. 5,830,960; 5,837,790; 6,248,849; and 6,387,995, each incorporated herein by reference (available commercially under the tradename of POLYTRAP® 7603 from AMCOL International, Arlington Heights, Ill.). These adsorbent polymeric microparticles also can be modified after incorporation of an active compound to further affect the rate of release of the active compound, as set forth in U.S. Pat. No. 6,491,953, incorporated herein by reference.

Another class of adsorbent polymeric microparticles prepared by a precipitation polymerization technique is disclosed in U.S. Pat. Nos. 4,962,170; 4,948,818; and 4,962,133, each incorporated herein by reference, and available commercially under the tradename of POLYTRAP by AMCOL International (INCI name of lauryl methacrylate/glycol dimethacrylate crosspolymer). An additional adsorbent polymeric microparticle has been developed wherein the active agent is added during the production of the polymeric microparticles, as disclosed in U.S. Patent Re. 33,429, incorporated herein by reference and sold under the tradename of MACROBEAD by AMCOL International (INCI name of lauryl methacrylate/glycol dimethacrylate crosspolymer). Other adsorbent polymers that are commercially available include, for example, MICROSPONGE® (INCI name of methyl methacrylate/glycol dimethylacrylate crosspolymer), available from AMCOL International, and Poly-HIPE polymers (e.g., a copolymer of 2-ethylhexyl acrylate, styrene, and divinylbenzene) available from Biopore Corporation, Mountain View, Calif.

### SUMMARY OF THE INVENTION

The present invention is directed to a polymeric microparticle delivery system capable of providing a visual cue to a consumer that a composition has been properly applied and/or has been applied for a sufficient time such that the composition performs its intended function. The polymeric microparticles are loaded with visual indicator, e.g., a dye or a pigment, either during the process of manufacturing the delivery system, i.e., during the synthesis of the microparticles, or after the microparticles have been prepared as a postsynthesis addition.

The polymeric microparticles are designed to prevent a premature release of the visual indicator into the composition during storage. However, after the composition has been applied, to a substrate, either animate or inanimate, the microparticles are degraded, e.g., fractured and/or fragmented, into smaller pieces through the mechanical energy provided by the consumer, thereby releasing the visual indicator and changing the color of the composition or treated substrate during and/or after application to a substrate.

In addition to the visual indicator, additional active and/or esthetic compounds also can be incorporated into the polymeric microparticles, or directly into the composition separate from the microparticles, for delivery during application of the composition. These microparticle delivery systems can be used in rinse-off compositions, such as a toothpaste, body wash, hand wash, shampoo, and hard surface cleaners, and in leave-on compositions, such as hand and body lotions, self-tanning products, and lip care products.

These and other aspects and novel features of the present invention will become apparent from the following detailed description of the preferred embodiments.

### DETAILED DESCRIPTION OF THE PREFERRED EMBODIMENTS

The present compositions comprise a delivery system comprising polymeric microparticles incorporating a visual indicator, such as a pigment or a dye, for inclusion in a composition. When the composition is applied to a substrate, the microparticles fragment and/or fracture, such that the composition and/or the substrate develops a color or undergoes a color transition, which indicates to the consumer that the composition has been properly applied. The microparticles also can provide a gentle cleaning action. In addition to containing a visual indicator, the delivery system also can incorporate an active agent to perform a predetermined or desired function on a treated substrate. The optional active agent can be added either during the polymerization reaction or in a post-reaction loading step. For example, the delivery system can be incorporated into a surfactant-containing composition, such as hand wash, body wash, shampoo, hard surface cleaner, and toothpaste products. The composition can be in the form of an emulsion, either water-in-oil or oil-in-water, an aqueous or nonaqueous gel, a dispersion, or a powder.

Polymeric microparticles are a delivery system often used in personal care and pharmaceutical formulations to extend release of an active ingredient, to protect the active ingredient from decomposition in a composition, and/or to enable formulation of the active ingredient into a composition due to difficulties, such as solubility or formulation esthetics.

Polymeric microparticle delivery systems comprise discrete, free-flowing particles which can absorb, adsorb, entrap, or otherwise retain an agent in a polymeric matrix. Such microparticles can provide a controlled release of the agent over time either by rupture of the microparticle, whereby the active agent is released when sufficient pressure or shearing action is applied to the microparticle, or the microparticle may be semipermeable or porous which allows the active agent to diffuse from the particle. Additionally, the microparticle delivery system can deliver multiple agents in addition to itself.

The term “polymeric microparticle delivery system” encompasses microparticles and microcapsules generally, which are a well-known form of polymeric beads formed by emulsion polymerization, precipitation polymerization, and other methods. Absorbent polymeric microparticles useful in the present invention have an ability to absorb several times their weight of a liquid compound.

The polymeric microparticles used in the present delivery system can be prepared either by suspension polymerization or by precipitation polymerization. After synthesis of the microparticles is complete, the microparticles are processed to remove residual monomers and/or solvents used in the manufacturing process.

In one example, the adsorbent polymer microparticles prepared by a suspension polymerization technique, e.g., POLY-PORE® E200, are a highly porous and highly crosslinked polymer in the form of open (i.e., broken) spheres and sphere sections characterized by a mean unit particle size of about 0.5 to about 3,000 microns, preferably about 0.5 to about 300 microns, more preferably about 0.5 to about 100 microns, and most preferably about 0.5 to about 80 microns. A significant portion of the spheres is about 20 microns in diameter.

The polymeric microparticles are oil and water adsorbent, and have an extremely low bulk density of about 0.008 gm/cc to about 0.1 gm/cc, preferably about 0.009 gm/cc to about 0.07 gm/cc, and more preferably about 0.0095 gm/cc to about 0.04-0.05 gm/cc. The microparticles are capable of holding and releasing oleophilic (i.e., oil soluble or dispersible), as well as hydrophilic (i.e., water soluble or dispersible), compounds, individually, or both oleophilic and hydrophilic compounds simultaneously.

Another class of adsorbent polymer microparticle prepared by a suspension polymerization technique, e.g. MACROBEAD, is a porous and highly crosslinked polymer in the form of spheres characterized by mean unit size of about 20 to about 3,000 microns, preferably about 20 to about 1000 microns, and most preferably about 20 to about 800 microns.

The adsorbent polymer microparticles prepared by the suspension polymerization technique include at least two polyunsaturated monomers, preferably allyl methacrylate and ethylene glycol dimethacrylate, and, optionally, a polyunsaturated and monounsaturated monomer mixture, such as lauryl methacrylate and ethylene glycol dimethacrylate. The microparticles are characterized by being open to their interior, due either to particle fracture upon removal of a porogen after polymerization or to subsequent milling or to the addition of a non-volatile porogen during the synthesis process. The microparticles have a mean unit diameter of less than about 2000 microns, preferably less than about 1000 microns.

Preferred polymeric microparticle delivery systems comprise a copolymer of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, and divinylbenzene, and mixtures thereof.

Specific polymeric microparticles useful in the present invention can be the previously described POLY-PORE® E200, POLYTRAP® 7603, POLYTRAP® 6603, MICROSPONGE® 5640, MACROBEAD® 7100, or Poly-HIPE particles, for example. A visual indicator and optional active agent are loaded onto such microparticles to provide a delivery system of the present invention. A visual indicator can be loaded onto such microparticles in an amount to provide microparticles containing 0.01% to about 50 wt. %, preferably about 0.05% to about 40 wt. %, and most preferably about 0.1% to about 20 wt. %, of the visual indicator, by weight of the loaded microparticles. The loaded microparticles typically are incorporated into a composition in an amount to provide about 0.5% to about 40%, preferably about 2% to about 30%, and typically about 1% to about 20%, by weight, of loaded polymeric microparticles in the composition.

To provide a delivery system for a visual indicator and an optional active agent, the visual indicator and optional active agent are incorporated, or loaded, onto or into the microparticles. As used herein, the term “loaded microparticle” refers to a microparticle having a visual indicator and an optional active agent added thereto. Loading of the active ingredient includes one or more of impregnating, imbedding, entrapping, absorbing, and adsorbing of a visual indicator and an optional active ingredient into or onto the polymeric microparticles. Loading of the active agent also can be referred to as an “entrapment” The term entrapment refers to a physical loading of the visual indicator and optional active ingredient onto the microparticles. Loading can be accomplished, for example, by spraying or adding the visual indicator and optional active agent directly to the microparticles in a manner such that a homogeneous distribution of the visual indicator and optional active agent on the microparticles is achieved.

Alternatively, the visual indicator and/or the optional active agent first can be dissolved in a suitable solvent, then the resulting solution is sprayed or added to the microparticles. The solvent then is removed by heating, vacuum, or both. As previously stated, two or more different types of materials can be added to the microparticles, wherein one of the materials is a visual indicator and the other material is an active agent and/or a material used to modify the release rate of compounds from the microparticles and/or to protect compounds loaded in the microparticles from reacting or otherwise interacting with other ingredients contained in the final composition. These release modifying or protective materials can be added directly to the microparticles in a molten state, or first dissolved in a suitable solvent, sprayed onto the microparticles followed by removal of the solvent from the delivery system. Alternatively, the visual indicator and optional active agent can be mixed with a release modifying or protective material, then loaded on a microparticle delivery system.

Another method of loading the visual indicator onto the polymeric microparticles is to add the visual indicator to the monomer mixture before polymerization. The visual indicator is added in such a way that it is uniformly dispersed in the monomer mixture. Additionally, an optional active agent can be added to monomer mixture, together with the visual indicator, before polymerization. The optional active agent preferably is soluble in or miscible with the monomer mixture. The resulting monomer mixture then is added to either the suspension or polymerization mixture for subsequent polymerization.

After loading the visual indicator and optional active agent on the microparticles, an optional barrier layer can be applied to the loaded microparticles to prevent rapid diffusion of compounds from the microparticles, and to protect the loaded compounds from the surrounding environment until application.

One approach for adding a visual indicator to the polymeric microparticles is to add a visual indictor, e.g., dye or pigment, to the microparticles after synthesis of the microparticles has been completed. In this approach, the visual indicator is incorporated into the microparticles either by dissolving or dispersing a visual indicator in a suitable solvent contacting the solvent with the dissolved or dispersed visual indicator with the microparticles, then removing the solvent under vacuum, by heating, or a combination thereof. The visual indicator can be loaded onto the microparticles in an amount of about 0.01% to about 80 wt. %, depending on the nature of the visual indicator, and the desired degree of color formation. In a more preferred loading, the visual indicator is loaded onto the microparticles in an amount of about 0.05% to about 70 wt. %, and in an even more preferred embodiment, in an amount of about 0.1% to about 60 wt. %.

The loaded microparticles can be added directly to a composition. In some embodiments, a need also may exist to ensure that leakage of the visual indicator into the formulation is avoided. In this case, a barrier layer can be applied to the loaded microparticles by adding a waxy material to the loaded microparticles.

A number of different waxy materials can be used as a barrier layer, including, but not limited to, low melting alcohols (C₈ through C₂₀) and fatty alcohols ethoxylated with one to three moles of ethylene oxide. Examples of fatty alcohols and alkoxylated fatty alcohols include, but are not limited to, behenyl alcohol, caprylic alcohol, cetyl alcohol, cetaryl alcohol, decyl alcohol, lauryl alcohol, isocetyl alcohol, myristyl alcohol, oleyl alcohol, stearyl alcohol, tallow alcohol, stearety-2, ceteth-1, cetearth-3, and laureth-2. Additional fatty alcohols and alkoxylated alcohols are listed in the International Cosmetic Ingredient Dictionary and Handbook, Tenth Edition, Volume 3, pages 2127 and pages 2067-2073 (2004), incorporated herein by reference.

Another class of materials that can be used in a barrier layer is the C₈ to C₁₂ fatty acids, including, but not limited to, stearic acid, capric acid, behenic acid, caprylic acid, laurie acid, myristic acid, tallow acid, oleic acid, palmitic acid, isostearic acid, and additional fatty acids listed in the International Cosmetic Ingredient Dictionary and Handbook, Tenth Edition, Volume 3, pages 2126-2127 (2004), incorporated herein by reference.

The barrier material also can be a hydrocarbon, like mineral oil, 1-decene dimmer, polydecene, paraffin, petrolatum, vegetable-derived petrolatum, or isoparaffin. Another class of barrier materials is waxes, like mink wax, carnauba wax, and candelilla wax, for example, and synthetic waxes, like silicone waxes, polyethylene, and polypropylene, for example.

Fats and oils can be useful barrier material agents, which include, for example, but are not limited to, lanolin oil, linseed oil, coconut oil, olive oil, menhaden oil, castor oil, soybean oil, tall oil, rapeseed oil, palm oil, and neatsfoot oil, and additional fats and oils listed in the International Cosmetic Ingredient Dictionary and Handbook, Tenth Edition, Volume 3, pages 2124-2126 (2004). Other useful classes of barrier materials include a water-insoluble ester having at least 10 carbon atoms, and preferably 10 to about 32 carbon atoms. Numerous esters are listed in International Cosmetic Ingredient Dictionary and Handbook, Tenth Edition, Volume 3, pages 2115-2123 (2004).

The waxy material can be present on the loaded microparticles in an amount of about 5% to about 70%, based on the weight of the loaded microparticles. In a more preferred embodiment, the ratio of the waxy material to the delivery system is about 10% to about 50%, and even more preferably about 20% to about 50 wt. %, based on the weight of the loaded microparticles.

In an alternate embodiment, a visual indicator is incorporated into an oil phase of a suspension polymerization process. In this embodiment, the visual indicator is soluble or dispersible in the hydrocarbon solvent used in the manufacture of the microparticles. The visual indicator also is inert with respect to the initiator used for the polymerization, and with respect to the free radical polymer chains generated during the polymerization reaction. An active agent, such as dimethicone or an ester solvent suitable for cosmetic, oral care, home care, or pharmaceutical applications, also can be a portion of the hydrocarbon solvent phase. This active agent, therefore, is included as a component of the microparticle delivery system.

The visual indicator can be any compound that is safe for use in personal care, oral care, home care, and pharmaceutical compositions. The visual indicator also is capable of temporarily imparting a color to a substrate after application of a composition containing a delivery system of the present invention to the substrate.

The visual indicator can be, for example, a dye, a pigment, a pH indicator, a natural food coloring, a fluorescent compound, or a mixture thereof. Typically, the visual indicator is a pigment or a dye. As used herein, the term “pigment” includes true pigments (i.e., colorants that precipitate as they are manufactured), toners (i.e., colorants produced by precipitating a water soluble dye as a metal salt), and lakes (i.e., nonsoluble colorants produced by adsorbing a water soluble dye onto an insoluble substrate). As used herein, the term “dye” includes, but is not limited to, a temporary dye or a semipermanent dye. Permanent dyes typically are avoided because the color generation or change may last too long for consumer acceptance. A preferred dye is a temporary dye, which has a minimal affinity for a substrate, and often are used for a specific event or occasion. The identity of the temporary dye and the semipermanent dye is not limited, and can be any temporary or semipermanent dye known or used in the art. Dyes useful for loading in the polymeric microparticles can be a direct dye, a disperse dye, an acid dye, a basic dye, or mixtures thereof.

Nonlimiting examples of pigments include, D & C Blue No. 1 Aluminum Lake, D & C Green No. 3 Aluminum Lake, D & C Orange No. 4 Aluminum Lake, D & C Orange No. 5 Aluminum Lake, D & C Orange No. 5 Zirconium Lake, D & C Orange No. 10 Aluminum Lake, D & C Orange No. 17 Lake, D & C Red No. 3 Aluminum Lake, D & C Red No. 4 Aluminum Lake, D & C Red No. 6 Aluminum Lake, D & C Red No. 6 Barium Lake, D & C Red No. 6 Barium/Strontium Lake, D & C Red No. 6 Potassium Lake, D & C Red No. 6 Strontium Lake, D & C Red No. 7 Aluminum Lake, D & C Red No. 7 Barium Lake, D & C Red No. 7 Calcium Lake, D & C Red No. 7 Calcium/Strontium Lake, D & C Red No. 7 Zirconium Lake, D & C Red No. 8 Sodium Lake, D & C Red No. 9 Aluminum Lake, D & C Red No. 9 Barium Lake, D & C Red No. 9 Barium/Strontium Lake, D & C Red No. 9 Zirconium Lake, D & C Red No. 19 Aluminum Lake, D & C Red No. 19 Barium Lake, D & C Red No. 19 Zirconium Lake, D & C Red No. 21 Aluminum Lake, D & C Red No. 21 Zirconium Lake, D & C Red No. 27 Aluminum Lake, D & C Red No. 27 Barium Lake, D & C Red No. 27 Calcium Lake, D & C Red No. 27 Zirconium Lake, Red 28 Aluminum Lake, D & C Red No. 30 Lake, D & C Red No. 31 Calcium Lake, D & C Red No. 33 Aluminum Lake, D & C Red No. 34 Calcium Lake, D & C Red No. 36 Lake, D & C Red No. 40 Aluminum Lake, D & C Yellow No. 5 Aluminum Lake, D & C Yellow No. 5 Zirconium Lake, D & C Yellow No. 6 Aluminum Lake, D & C Yellow No. 7 Zirconium Lake, D & C Yellow No. 10 Aluminum Lake, Ext. D & C Yellow No. 7 Aluminum Lake, FD & C Blue No. 1 Aluminum Lake, FD & C Red No. 3 Aluminum Lake, FD & C Red No. 4 Aluminum Lake, FD & C Yellow No. 5 Aluminum Lake, FD & C Yellow No. 6 Aluminum Lake, Ultramarine Blue, Ultramarine Green, Ultramarine Pink, Ultramarine Red, Ultramarine Violet, and mixtures thereof.

A pigment can be used alone, or in combination with a dye. The dye can be one or a mixture of temporary dyes, a mixture of semipermanent dyes, or a mixture of temporary and semipermanent dyes.

Temporary dyes useful in the first composition typically are monoazo, diazo, polyazo, quinoline, anthraquinone, triphenylmethane, or naphthoquinoneimine dyes. Exemplary temporary dyes include, but are not limited to Acid Yellow 3, Acid Orange 7, Acid Orange 24, Direct Red 80, Food Red 1, Acid Red 33, Acid Violet 43, Acid Blue 9, Acid Green 25, Direct Black 57, Basic Yellow 57, Basic Yellow 87, Basic Red 76, Basic Red 51, Basic Blue 99, Basic Brown 16, Basic Brown 17, Acid Yellow 1, Acid Yellow 9, Disperse Yellow 3, Disperse Yellow 1, Basic Violet 1, Basic Violet 3, FD&C Blue No. 1, FD&C Red No. 4, FD&C Yellow No. 5, FD&C Yellow No. 6, FD&C Green No. 3, FD&C Red No. 40, D&C Green No. 3, D&C Orange No. 5, D&C Red No. 6, D&C Red No. 7, D&C Red No. 22, D&C Red No. 28, D&C Red No. 30, D&C Red No. 33, D&C Red No. 34, D&C Red No. 36, D&C Orange No. 4, D&C Yellow No. 10, D&C Green No. 8, Ext. D&C Violet No. 2, Ext. D&C Yellow No. 7, D & C Blue No. 4, D & C Brown No. 1, D & C Green No. 5, D & C Green No. 6, D & C Orange No. 10, D & C Orange No. 11, D & C Orange No. 17, D & C Red No. 8, D & C Red No. 9, D & C Red No. 17, D & C Red No. 19, D & C Red No. 21, D & C Red No. 27, D&C Red No. 31, D & C Violet No. 2, D & C Yellow No. 7, D & C Yellow No. 8, D & C Yellow No. 11, FD & C Red No. 3, salts thereof, and mixtures thereof.

Semipermanent dyes typically are a nitrophenylenediamine or a nitroaminophenolic ether, illustrated as compounds (1) and (2), respectively, wherein R¹ through R⁴ typically are hydrogen, alkyl, hydroxyalkyl, or polyhydroxyalkyl.

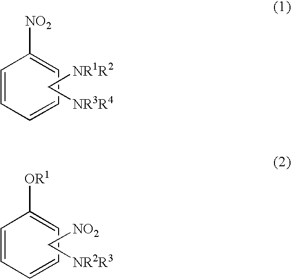

The semipermanent dye also can be an aminoanthraquinone.

Nonlimiting examples of semipermanent dyes include 2-hydroxyethyl picramic acid, 4-nitro-m-phenylenediamine, HC Yellow 10, 4-nitro-o-phenylenediamine, HC Blue 7, HC Yellow 2, HC Yellow 5, 2-nitro-p-phenylenediamine, HC Red 14, HC Red 10, HC Red 11, HC Red 1, HC Red 3, HC Red 13, N,N-bis(2-hydroxyethyl)-2-nitro-p-phenylenediamine, HC Violet 2, HC Blue 2, 2-amino-4-nitrophenol, 2-amino-5-nitrophenol, HC Yellow 4, HC Yellow 9, 2-nitro-5-glycerylmethylaniline, 2-amino-3-nitrophenol, 2-nitro-N-hydroxyethyl-p-anisidine, HC Orange 2, HC Orange 3, 4-amino-3-nitrophenol, 4-hydroxypropylamino-3-nitrophenol, 3-nitro-p-hydroxyethylaminophenol, Disperse Red 15, Disperse Red 11, Disperse Violet 1, Disperse Violet 4, Disperse Blue 3, BC Blue 14, HC Yellow 6, HC Yellow 15, HC Yellow 1, HC Orange 1, HC Yellow 7, Disperse Black 9, salts thereof, and mixtures thereof.

Additional temporary and semipermanent dyes useful alone, or in any combination, or as a salt, in first hair color revitalizing composition include, but are not limited to, Acid Black 131, Acid Blue 62, Acid Orange 3, Acid Red 35, Acid Red 52, 2-amino-6-chloro-4-nitrophenol, 4-amino-2-hydroxytoluene, 2-amino-3-nitrophenol, m-aminophenol, o-aminophenol, p-aminophenol, Basic Blue 6, Basic Blue 9, Basic Blue 41, Basic Brown 4, Basic Red 22, Basic Yellow 11, N,N-bis(2-hydroxyethyl)-p-phenylenediamine sulfate, bismuth citrate, 2-chloro-p-phenylenediamine, 2,4-diaminodiphenylamine, 4,4′-diaminodiphenylamine, 2,4-diaminophenol, 2,4-diaminophenoxyethanol, 2,6-diaminopyridine, N,N-diethyl-m-aminophenol, N,N′-dimethyl-N-hydroxyethyl-3-nitro-p-phenylenediamine, dimethyl o-toluidine, 2,5-dinitrophenol, Direct Black 51, Direct Red 23, Direct Red 81, Direct Yellow 48, Direct Violet 48, Direct Yellow 12, Disperse Blue 1, HC Blue No. 4, HC Blue No. 5, HC Yellow No. 3, HC Yellow No. 5, henna, hydroxybenzomorpholine, N-hydroxyethyl-2-amino-4-hydroxytoluene sulfate, 2-hydroxyethylamino-5-nitroanisole, lead acetate, N-methoxyethyl-p-phenylenediamine, 2-methoxyethyl-p-phenylenediamine, 2-methoxyethyl-p-phenylenediamine, 2-methoxy-p-phenylenediamine, 4-methoxytoluene-2,5-diamine, 3-methylamino-4-nitrophenoxyethanol, p-methylaminophenol, 2-methyl-5-hydroxyethylaminophenol, N-methyl-2-nitro-p-phenylenediamine, 2-methylresorcinol, 1,5-naphthalenediol, 2,3-naphthalenediol, 2,7-naphthalenediol, 1-naphthol, 2-naphthol, 3-nitro-4-aminophenoxyethanol, 2-nitro-N-hydroxyethyl-p-anisidine, m-phenylenediamine, p-phenylenediamine, phenyl methyl pyrazolone, N-phenyl-p-phenylenediamine, phloroglucinol, picramic acid, Pigment Blue 15, Pigment Blue 15:2, Pigment Green 7, Pigment Red 112, Pigment Violet 19, Pigment Yellow 1, Pigment Yellow 3, Pigment Yellow 12, Pigment Yellow 13, Pigment Yellow 73, pyrocatechol, pyrogallol, resorcinol, sodium picramate, sodium sulfanilate, Solvent Black 3, Solvent Blue 35, Solvent Red 24, Solvent Yellow 44, toluene-2,5-diamine, toluene-3,4-diamine, salts thereof, and mixtures thereof. Further dyes useful in the delivery system are disclosed in U.S. Pat. Nos. 6,746,492 and 6,835,018, each incorporated herein by reference.

Additional dyes and pigments useful as visual indicators are disclosed in Kirk-Othmer Encyclopedia of Chemical Technology, 4th Ed., Vol. 6, pages 892-944 (1991); U.S. Pat. No. 4,568,534; U.S. Pat. No. 4,150,106; U.S. Pat. No. 6,419,902; U.S. Patent Publication No. 2006/0115435, each incorporated herein by reference.

In addition a pigment and/or dye, the visual indicator can be an acid-base indicator, used alone or together with a pigment, a dye, a second acid-base indicator. Suitable acid-base indicators used in the present invention generally are colored under basic conditions and change color or fade to clear in non-basic conditions. Acid-base indicators which are colored on the alkaline pH side (pH>7) and turn clear on acidic pH side (pH<7) are most useful. Typically, the acid-base indicators are colored at pH between about 9 and 10, and turn clear at pH between about 6 and 8.

Useful acid-base indicators, or pH indicators, that can be loaded onto the polymeric microparticles include, but are not limited to, chlorophenol red, phenolphthalein, FD & C Blue No. 1, D & C Yellow No. 10, picric acid, matius yellow, 2,6-dinitrophenol, 2,4-dinitrophenol, phenoacetolin, 2,5-dinitrophenol, isopicramic acid, o-nitrophenol, m-nitrophenol, p-nitrophenol, 6,8-dinitro-2,4-(1H,3H)quinazolinedione, nitroamine, ethyl bis(2,4-dinitrophenyl)-acetate, 2,4,6-trinitrotoluene, 1,3,5-trinitrobenzene, 2,4,6-tribromobenzoic acid, 2-(p-dimethylaminophenyl)azopyridine, metanil yellow, p-methyl red, 4-phenylazodiphenylamine, benzopurpurin 4B, tropaeolin 00, fast garnet GBC base, alizarin yellow R, benzyl orange, m-methyl red, 4-(m-tolyl)-azo-N,N-dimethyl-aniline, oil yellow II, methyl orange, ethyl orange, hessian purple N, congo red, N-phenyl-1-naphthyl-aminoazobenzene-p-sulfonic acid, 4-(4′-dimethylamino-1 ′-naphthyl)-azo-3-methoxy-benzenesulfonic acid, p-ethoxychrysoidine, α-naphthyl red, chrysoidine, 1-naphthylaminoazobenzene-p-sulfonic acid, methyl red, 2-(p-dimethylaminophenylopyridine, ethyl red, propyl red, N-phenyl-1-naphthyl-aminoazo-o-carboxybenzene, nitrazol yellow, brilliant yellow, brilliant yellow S, orange II, propyl-o-naphthyl orange, orange I, orange IV, hessian, Bordeaux, diazo violet, a-naphthol violet, alizarin yellow GG, chrome orange GR, sulfone acid blue R, lanacyl violet BF, tropaeolin O, orange G, crystal violet, methyl violet B, malachite green, brilliant green, ethyl violet, methyl violet 6B, ethyl/methyl green, basic fuchsine, acid, fuchsine, patent blue V, alkali blue, aniline blue, o-naphthol benzein, pentamethoxy red, hexamethoxy red, tetrabromophenolphthalein ethyl ester K salt, tetraiodophenolsulfophthlein, bromochlorophenol blue, bromocresol green, chlorocresol green, bromocresol purple, sulfonaphthyl red, bromophenol red, dibromophenol-tetrabromophenolsulfophthlein, bromothymol blue, aurin, phenol red, o-cresol benzein, o-cresol red, α-naphtholphthlein, m-cresol purple, p-xylenol blue, thymol blue, phenoltetrachlorophthlein, o-cresolphthalein, α-naphtholbenzein, phenoltetraiodophthlein, thymolphthlein, eosin Y, erythrosine B, erythrosine, galleon, brilliant cresyl blue, resazurin, lacmoid, litmus, azolitmus, azolitmin, neutral red, nile blue 2B, nile blue A, hematoxylin, quinaldine red, pinachrome, indooxine, quinoline blue, bis-5-bromovanillidenecyclohexanone, bis-(2t-hydroxystyryl)ketone, curcumin, bis-(4-hydroxy-3-ethoxybenzylidene)-cyclohexanone, thiazole yellow G, alizarin blue B, alizarin red S, carminic acid, alizarin orange, alizarin, rufianic acid, rufianic blue, alizarin blue SWR, indigocai wine, and mixtures thereof. Additional acid-base indicators are disclosed in U.S. Patent Publication No. 2006/0222601, incorporated herein by reference.

With a suitable selection of acid-base indicators, it is possible to produce any color. The acid-base indicators often are used in the form of a salt, such as a sodium salt generated by reacting the indicator with sodium hydroxide. Additionally, combinations of two or more acid-base indicators may be used.

The visual indicator also can be a natural food color, for example, carmine, carotene, annatto, turmeric, curcumin, and other natural food colors known in the art.

The visual indicator further can be a fluorescent compound, for use alone, or with one or more of a pigment, dye, acid-base indicator, and natural food color. Examples of fluorescent compounds include, but are not limited to, a distyrylbiphenyl derivative, stilbene or a stilbene derivative, a pyralozine derivative, or a coumarin derivative. The optional brightener typically is a derivative of stilbene or 4,4r-diaminostilbene, biphenyl, a 5-membered heterocycle, e.g., triazole, oxazole, or imidazole, or a 6-membered heterocycle, e.g., a coumarin, a naphthalamide, or an s-triazine.

The optical brighteners are available under a variety of tradenames, such as TINOPAL®, LEUCOPHOR®, and CALCOFLUOR®. Specific fluorescent compounds include, but are not limited to, TINOPAL® 5BM, CALCOFLUOR® CG, and LEUCOPHOR® BSB.

In accordance with an important feature of the present invention, an optional active agent present in the delivery system can be any of a wide variety of compounds, either water soluble or oil soluble. A composition containing a present delivery system, therefore, can be applied to a substrate, like the skin, and the active agent then performs its intended function.

The active agent can be a water-soluble or water-dispersible compound, i.e., is hydrophilic. The active agent also can be oil soluble or oil dispersible, i.e., is hydrophobic. In other embodiments, the active agent is a mixture of compounds, either all hydrophilic, all oleophilic, or a mixture of hydrophilic and oleophilic compounds.

The optional active agent, therefore, can be one of, or a mixture of, a cosmetic compound, a medicinal compound, or any other compound that is useful upon topical application to the skin, teeth, or hair. Such active agents include, but are not limited to, skin care compounds, antibacterial compounds, antifungal compounds, antiacne agents, antiinflammatory compounds, pharmaceuticals, topical anesthetics, sunscreens, an insect repellant, and other cosmetic and medicinal topically effective compounds. The quantities of such optional active agents present in the polymeric microparticles are sufficient to perform their intended function, without adversely affecting the benefits of other ingredients present in the composition.

For example, a skin conditioner can be the optional active agent in a delivery system of the present invention. Skin conditioners include, but are not limited to, humectants, such a fructose, glucose, glycerin, propylene glycol, glycereth-26, mannitol, and urea, pyrrolidone carboxylic acid, hydrolyzed lecithin, coco-betaine, cysteine hydrochloride, glucamine, PPG-15, sodium gluconate, potassium aspartate, oleyl betaine, thiamine hydrochloride, sodium laureth sulfate, sodium hyaluronate, hydrolyzed proteins, hydrolyzed keratin, amino acids, amine oxides, water-soluble derivatives of vitamins A, E, and D, silicones, amino-functional silicones, ethoxylated glycerin, alpha-hydroxy acids and salts thereof, triglycerides, fatty oil derivatives, such as PEG-24 hydrogenated lanolin, almond oil, grape seed oil, and castor oil, and mixtures thereof. Numerous other skin conditioners are listed in the CTFA Cosmetic Ingredient Handbook, Tenth Ed., T. E. Gottshalck et al, ed., The Cosmetic, Toiletry and Fragrance Association (2004), (hereafter CTFA Handbook), pages 2392-2395, incorporated herein by reference.

If the composition is intended to be a sunscreen, then compounds such as benzophenone-3, trihydroxycinnamic acid and salts, tannic acid, uric acids, quinine salts, dihydroxy naphtholic acid, an anthranilate, diethanolamine methoxycinnamate, p-aminobenzoic acid, phenylbenzimidazole sulfonic acid, PEG-25, p-aminobenzoic acid, or triethanolamine salicylate can be used as the active agent.

Further, sunscreen compounds such as dioxybenzone, ethyl 4-[bis(hydroxypropyl)]aminobenzoate, glyceryl aminobenzoate, homosalate, methyl anthranilate, octocrylene, octyl methoxycinnamate, octyl salicylate, oxybenzone, padimate O, red petrolatum, titanium dioxide, 4-menthylbenzylidene camphor, benzophenone-1, benzophenone-2, benzophenone-6, benzophenone-12, isopropyl dibenzoyl methane, butyl methoxydibenzoylmethane, octocrylene, or zinc oxide can be used as the active agent. Other sunscreen compounds are listed in CTFA Handbook, pages 2397-2399, incorporated herein by reference.

Similarly, active agents, like antifungal compounds, antibacterial compounds, anti-inflammatory compounds, topical anesthetics, skin rash, skin disease, and dermatitis medications, and anti-itch and irritation-reducing compounds can be used as the active agent in compositions of the present invention. For example, analgesics such as benzocaine, dyclonine hydrochloride, aloe vera, and the like; anesthetics such as butamben picrate, lidocaine hydrochloride, xylocaine, and the like; antibacterials and antiseptics, such as povidone-iodine, polymyxin b sulfate-bacitracin, zinc-neomycin sulfate-hydrocortisone, chloramphenicol, ethylbenzethonium chloride, erythromycin, and the like; antiparasitics, such as lindane; essentially all dermatologicals, like acne preparations, such as benzoyl peroxide, erythromycin, clindamycin phosphate, 5,7-dichloro-8-hydroxyquinoline, and the like; antiinflammatory agents, such as alclometasone dipropionate, betamethasone valerate, and the like; burn relief ointments, such as o-amino-p-toluenesulfonamide monoacetate, and the like; depigmenting agents, such as monobenzone; dermatitis relief agents, such as the active steroid amcinonide, diflorasone diacetate, hydrocortisone, and the like; diaper rash relief agents, such as methylbenzethonium chloride, and the like; emollients and moisturizers, such as lanolin oil, petrolatum, mineral wax, and the like; fungicides, such as butocouazole nitrate, haloprogin, clotrimazole, and the like; herpes treatment drugs, such as O-[(2-hydroxymethyl)-methyl]guanine; pruritic medications, such as alclometasone dipropionate, betamethasone valerate, isopropyl myristate MSD, and the like; psoriasis, seborrhea, and scabicide agents, such as anthralin, methoxsalen, coal tar, and the like; steroids, such as 2-(acetyloxy)-9-fluoro-1′,2′,3′,4′-tetrahydro-11-hydroxypregna-1,4-dieno-[16,17-b]naphthalene-3,20-dione and 21-chloro-9-fluoro-1′,2′,3′,4′-tetrahydro-11b-hydroxypregna-1,4-dieno-[16,17-b]naphthalene-3,20-dione. Any other medication capable of topical administration, like skin bleaching agents, skin protestant, such as allantoin, and antiacne agents, such as salicylic acid, also can be incorporated in a composition of the present invention in an amount sufficient to perform its intended function. Other topically active compounds are listed in Remington's Pharmaceutical Sciences, 17th Ed., Merck Publishing Co., Easton, Pa. (1985), pages 773-791 and pages 1054-1058 (hereinafter Remington's), incorporated herein by reference.

The optional active agent also can be an oral care compound. A variety of oral care compounds can be incorporated into the polymeric microparticles. The oral care compounds include, but are not limited to:

(a) antibacterials, such as a halogenated diphenyl ethers, e.g., 2′,4,4′-trichloro-2-hydroxy-diphenyl ether, known under the trade name triclosan, and 2,2′-dihydroxy-5,5′-dibromo-diphenyl ether; 2,2′-methylenebis-4-4-chloro-6-bromo-phenol); halogenated salicylanilides; halogenated carbanilides; sodium tripolyphosphate; cetyl pyridinium chloride; benzalkonium chloride; sodium hypochlorite; hexachlorophene; thymol; cresols; guaiacol; eugenol; creosote; copper sulphate; copper-(ethyl) maltol; zinc- and stannous salts, such as zinc citrate and sodium zinc citrate; stannous pyrophosphate; and sanguinarine extract;

(b) a caries prophylactic, such as a fluoride ion source like sodium fluoride, stannous fluoride, and sodium mono fluorophosphate; sodium chloride; and sodium bicarbonate;

(c) a tooth whitener, such as hydrogen peroxide, sodium percarbonate, sodium perborate, potassium peroxydiphosphate, and organic peracids;

(d) an antiplaque agent, such as a silicone polymer;

(e) an analgesic, such as codeine, aspirin, acetaminophen, propoxyphene, meperidine, and benzocaine;

(f) a flavor, such as spearmint oil, methyl salicylate, cinnamon oil, peppermint oil, clove oil, saccharin, thymol, menthol, and eucalyptus; and

(g) a surfactant, such as sodium lauryl sulfate.

The active agent also can be a plant extract on a natural oil. Nonlimiting plant extracts are those obtained from alfalfa, aloe vera, amla fruit, angelica root, anise seed, apple, apricot, artichoke leaf, asparagus root, banana, barberry, barley sprout, bee pollen, beet leaf, bilberry fruit, birch leaf, bitter melon, black currant leaf, black pepper, black walnut, blueberry, blackberry, burdock, carrot, cayenne, celery seed, cherry, chickwood, cola nut, corn silk, cranberry, dandelion root, elderberry, eucalyptus leaf, flax oil powder, ginger root, gingko leaf, ginseng, goldenrod, goldenseal, grape, grapefruit, guava, hibiscus, juniper, kiwi, kudzu, lemon, licorice root, lime, malt, marigold, myrrh, olive leaf, orange fruit, orange peel, oregano, papaya fruit, papaya leaf, passion fruit, peach, pear, pine bark, plum, pomegranate, prune, raspberry, rice bran, rhubarb root, rosemary leaf, sage leaf, spearmint leaf, St. John's wart, strawberry, sweet cloves, tangerine, violet herb, watercress, watermelon, willow bark, wintergreen leaf, witch hazel bark, yohimbe, and yucca root.

The polymeric microparticles also can contain cleansing agents for animate and inanimate surfaces, such as an alcohol or a surfactant. The surfactant can be an anionic surfactant, a cationic surfactant, a nonionic surfactant, or a compatible mixture of surfactants. The surfactant also can be an ampholytic or amphoteric surfactant, which have anionic or cationic properties depending on the pH of the composition.

Examples of anionic surfactants include, without limitation, soaps, alkyl sulfates, anionic acyl sarcosinates, methyl acyl taurates, N-acyl glutamates, acyl isethionates, alkyl phosphate esters, ethoxylated alkyl phosphate esters, alkyl sulfosuccinates, trideceth sulfates, protein condensates, mixtures of ethoxylated alkyl sulfates, and the like. Examples of anionic nonsoap surfactants include, without limitation, the alkali metal salts of an organic sulfate having an alkyl radical containing about 8 to about 22 carbon atoms and a sulfonic acid or sulfuric acid ester radical.

Examples of zwitterionic surfactants include, without limitation, derivatives of aliphatic quaternary ammonium, phosphonium, and sulfonium compounds, in which the aliphatic radicals can be straight chain or branched and wherein one of the aliphatic substituents contains an anionic water-solubilizing group, e.g., carboxyl, sulfonate, sulfate, phosphate, or phosphonate. Examples of amphoteric surfactants include, without limitation, derivatives of aliphatic secondary and tertiary amines in which the aliphatic radical can be straight chain or branched and wherein one of the aliphatic substituents contains about 8 to about 18 carbon atoms and one contains an anionic water solubilizing group, e.g., carboxyl, sulfonate, sulfate, phosphate, or phosphonate.

Examples of cationic surfactants include, without limitation, stearyldimethylbenzyl ammonium chloride; dodecyltrimethyl ammonium chloride; nonylbenzylethyldimethyl ammonium nitrate; and tetradecylpyridinium bromide.

Nonionic surfactants include, without limitation, compounds produced by the condensation or ethylene oxide groups with an organic hydrophobic compound, which may be aliphatic or alkyl aromatic in nature, for example, the polyethylene oxide condensates of alkyl phenols.

The compositions of the present invention also can include optional ingredients traditionally included in cosmetic, home care, medicinal, and other such compositions. These optional ingredients include, but are not limited to, fragrances, preservatives, antioxidants, detackifying agents, and similar types of compounds. The optional ingredients are included in the composition in an amount sufficient to perform their intended function.

### EXAMPLES

### Example 1

### Preparation of Blue Particles

A suspension polymerization was performed. Methocel K100 (5.55 g) was added to 962.8 g of deionized (DI) water, and the resulting mixture was stirred at room temperature (e.g., 20° C. to 25° C.). An organic phase, i.e., toluene (129 g), ethylene glycol dimethacrylate (EGDMA, 186 g), lauryl methacrylate (6.14 g), and hydrophobic Ultramarine Blue (LCW C9404 hydrophobic Ultramarine Blue (23.6 g), was admixed in a separate container for 30 minutes, at which time 0.6 g of Vazo 52 (DuPont) was added. The stirring speed of the main reactor was reduced to 128 rpm, then the organic phase was added into the water phase. The organic phase beaker was washed with deionized water (DI) water, the reaction was purged with an inert gas, and heating to between 73° C. and 81° C. was initiated. The reaction was maintained between 73° C. and 83° C. overnight. The resulting blue polymeric microparticles were filtered, then dried in a vacuum oven. The median particle size of the polymeric microparticles was about 600 microns.

### Example 2

### Preparation of Blue Particles with Dimethicone

A suspension polymerization was performed. Methocel K100 (5.6 g) was added to 1119.2 g of DI water, and the resulting mixture was stirred at room temperature. An organic phase, i.e., toluene (129 g), ethylene glycol dimethacrylate (EGDMA, 187 g), lauryl methacrylate (6.24 g), dimethicone (350 est, 3.84 g), dimethicone (10 cst, 12.4 g), and hydrophobic Ultramarine Blue (23.7 g), was admixed in a separate container for 30 minutes, at which time 0.5 g of Vazo 52 (DuPont) was added. The stirring speed of the main reactor was reduced to 128 rpm, then the organic phase was added into the water phase. The organic phase beaker was washed with DI water, the reaction mixture was purged with an inert gas, and heating to between 73° C. and 81° C. was initiated. The reaction was maintained between 73° C. and 83° C. overnight. The blue polymeric microparticles were filtered, then dried in a vacuum oven. The median particle size of the polymeric microparticles was about 400 microns.

### Example 3

### Preparation of Blue Particles with Dimethicone

A suspension polymerization was performed. Methocel K100 (5.9 g) was added to 1114.3 g of DI water, and the resulting mixture was stirred at room temperature. An organic phase, i.e., toluene (137 g), ethylene glycol dimethacrylate (EGDMA, 211 g), lauryl methacrylate (69.5 g), dimethicone (350 cst, 68 g), dimethicone (100 cst, 68 g), and hydrophobic Ultramarine Blue (25.2 g), was admixed in a separate container for 30 minutes, at which time 0.7 g of Vazo 52 (DuPont) was added. The stirring speed of the main reactor was reduced to 128 rpm, then the organic phase was added into the water phase. The organic phase beaker was washed with DI water, the reaction mixture was purged with an inert gas, and heating to between 73° C. and 81° C. was initiated. The reaction was maintained between 73° C. and 83° C. overnight. The blue polymeric particles were filtered, then dried in a vacuum oven. The median particle size of the polymeric microparticles was about 400 microns.

### Example 4

### Preparation of Blue Particles during Polymerization

A suspension polymerization was performed. Methocel K100 (2.55 g) was added to 1117.7 g of DI water, and the resulting mixture was stirred at room temperature. An organic phase, i.e., ethylene glycol dimethacrylate (EGDMA, 76.5 g), lauryl methacrylate (176.97 g), dimethicone (350 cst, 273.5 g), and hydrophobic Ultramarine Blue (50.3 g), was admixed in a separate container for about 60 minutes with a high mixer, at which time 0.6 g of Vazo 52 (DuPont) was added. The stirring speed of the main reactor was reduced to 125-135 rpm, then the organic phase was added to the water phase. The organic phase beaker was washed with DI water, the reaction mixture was purged with an inert gas, and heating to between 73° C. and 81° C. was initiated. The reaction was maintained between 73° C. and 83° C. overnight. The resulting blue polymeric particles were filtered, and then dried in a vacuum oven.

### Example 5

### Blue Particles with Sesame Seed Oil

A suspension polymerization was performed. Methocel K100 (1.99 g) was added to 1358.6 g of DI water, and the resulting mixture was stirred at room temperature. An organic phase, i.e., ethylene glycol dimethacrylate (EGDMA, 913 g), lauryl methacrylate (93.3 g), sesame seed oil (115.3 g), and Pigment Blue 15:3 (VIBRABLUE 15.3, Ciba Speciality, 37.0 g), was admixed in a separate container for about 60 minutes with a high mixer, at which time 0.51 g of Vazo 52 (DuPont) was added. The stirring speed of the main reactor was reduced to 125-135 rpm, then the organic phase was added to the water phase. The organic phase beaker was washed with DI water, the reaction mixture was purged with an inert gas, and heating to between 73° C. and 81° C. was initiated. The reaction was maintained between 73° C. and 83° C. overnight. The resulting blue polymeric particles were filtered, and then dried in a vacuum oven.

### Example 6

To 2 g of POLY-PORE® E200 was added 2 g of a molten mixture that contained 95 wt. % stearyl alcohol and 5 wt. % hydrophobically modified Ultramarine Blue. The final product was a pale blue color.

### Example 7

To 2 g of POLY-POR® E200 was added 2 g of a molten mixture of 90 wt. % stearyl alcohol and 10 wt. % hydrophobically modified Ultramarine Blue. The final concentration of visual indicator in the loaded microparticles was 5 wt. % pigment, with 45 wt. % stearyl alcohol also loaded onto the particles. The final product was a pale blue color.

### Example 8

### Color Stability

The microparticles of Example 3 were added to various cleanser products, and the resulting compositions were subjected to accelerated temperatures to mimic long term shelf life. In one test, the microparticles were added to SOFT SCRUB Lemon Cleanser, Dial Corporation (pH 10) and SOFT SCRUB Cleanser with bleach (pH 11) No bleeding of the color was noted in either composition after one month at 40° C. or after 1 week at 60° C. The microparticles also were added to Colgate Total toothpaste, which after storing for one month at 50° C. and after three months at 45° C., demonstrated no indication of pigment bleeding. The microparticles further were added to an Adidas clear shower gel, and after 40 days at 60° C., no color bleeding from the particles was observed.

### Example 9

### Stability of Color

The microparticles of Example 5 were added to a toothpaste base formulation. After one month at 60° C., no pigment bleeding from the particles was observed.

Examples 8 and 9 clearly show that a visual indicator loaded onto a polymer microparticle remains entrapped in the microparticles until application to a substrate. During application, the polymeric microparticles fracture to release the visual indicator. Compositions of the present invention, therefore, can be stored for extended time periods of one to several months, e.g., a year, even at elevated temperature of a about 40° C. to about 60° C., without a premature release of the visual indicator. According to common practice in the cosmetic industry these results that the products will be stable at ambient temperature for more than two years.

### Example 10

### Brushing Performance in a Toothpaste

To 9.82 grams of toothpaste base, was added 0.18 g of the blue microparticles of Example 5. The microparticles were mixed with toothpaste until they were uniformly distributed throughout the paste. The microparticles did not break down during this addition. The toothpaste/microparticles mixture was transferred to the back of a ceramic tile that was pre-wetted with water. A medium bristle toothbrush was wetted with water, then the toothpaste was brushed for 2 minutes during which time care was taken to make sure that all the toothpaste was brushed. At the end of 2 minutes, the toothpaste was transferred to a Petri dish. The Petri dish was inverted and the color of the toothpaste was analyzed by a color meter (Hunter Lab). The b* value of the color scale was −20.3.

Compositions of the present invention can be applied to an animate or an inanimate substrate. An animate substrate, for example, is skin or teeth of a mammal. An inanimate substrate, for example, is a hard surface, like a countertop or a bathroom fixture.

Obviously, many modifications and variations of the invention as hereinbefore set forth can be made without departing from the spirit and scope thereof and, therefore, only such limitations should be imposed as are indicated by the appended claims.

What is claimed is:

1. A composition comprising a microparticle delivery system, said delivery system comprising polymeric microparticles having a visual indicator incorporated therein.

2. The composition of claim 1 wherein the polymeric microparticles are highly crosslinked and are derived from methacrylate monomers, acrylate monomers, or mixtures thereof.

3. The composition of claim 1 wherein the polymeric microparticles comprise an ally methacrylate copolymer, an ethylene glycol dimethacrylate/allyl methacrylate copolymer, a lauryl methacrylate/ethylene glycol dimethacrylate copolymer, a methyl methacrylate/ethylene glycol dimethacrylate copolymer, and mixtures thereof.

4. The composition of claim 1 wherein the polymeric microparticles are selected from the group consisting of a copolymer of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, a divinylbenzene, and mixtures thereof.

5. The composition of claim 1 wherein the polymeric microparticles comprise a copolymer of allyl methacrylate and ethylene glycol dimethacrylate, a copolymer of ethylene glycol dimethacrylate and lauryl methacrylate, or a mixture thereof.

6. The composition of claim 5 wherein the polymeric microparticles comprise copolymer of ethylene glycol dimethacrylate and lauryl methacrylate.

7. The composition of claim 1 wherein the visual indicator comprises a dye, a pigment, an acid-base indicator, a natural food color, a fluorescent compound, or mixtures thereof.

8. The composition of claim 7 wherein the visual indicator comprises a pigment.

9. The composition of claim 7 wherein the visual indicator comprises a dye.

10. The composition of claim 7 wherein the visual indicator comprises an acid-base indicator.

11. The composition of claim 7 wherein the visual indicator comprises a natural food color.

12. The composition of claim 7 wherein the visual indicator comprises a fluorescent compound.

13. The composition of claim 1 wherein the visual indicator is loaded onto the microparticles during manufacture of the microparticles.

14. The composition of claim 1 wherein the visual indicator is loaded onto the microparticles after manufacture of the microparticles.

15. The composition of claim 1 wherein about 0.01% to about 50% of the visual indicator is loaded onto the microparticles, based on the weight of the loaded microparticles.

16. The composition of claim 1 wherein the composition comprises about 0.5% to about 40%, by weight, of the loaded microparticles.

17. The composition of claim 1 wherein the visual indicator is selected from the group consisting of Ultramarine Blue and Pigment Blue 15:3.

18. The composition of claim 1 wherein the polymeric microparticles further comprise an active agent.

19. The composition of claim 18 wherein the active agent comprises a compound selected from the group consisting of a pesticide, a drug, a therapeutic agent, a deodorant, a hormone, an analgesic, an anti-acne agent, a pharmaceutical, a skin conditioner, a skin protectant, a skin-soothing agent, an antioxidant, an insect repellant, a counterirritant, a vitamin, a plant extract, a steroid, an antibacterial compound, an antifungal compound, an anti-inflammatory compound, a topical anesthetic, an epidermal lipid replacement, an anti-aging compound, a sunscreen, a skin whitener, an optical brightener, an antiperspirant, a disinfectant, a sanitizes, a dermatitis or skin disease medication, a cleanser, an alcohol, and mixtures thereof.

20. The composition of claim 18 wherein the active agent is selected from the group consisting of benzocaine, dyclonine hydrochloride, aloe vera, butamben picrate, lidocaine hydrochloride, xylocaine, providone-iodine, polymyxin b sulfate-bacitracin, zinc-neomycin sulfate-hydrocortisone, chloramphenicol, ethylbenzethonium chloride, erythromycin, lindane, benzoyl peroxide, erythromycin benzoyl peroxide, clindamycin phosphate, 5,7-dichloro-8-hydroxyquinoline, alclometasone dipropionate, betamethasone valerate, o-amino-p-toluenesulfonamide monoacetate, monobenzone, amcinonide, diflorasone diacetate, hydrocortisone, methylbenzethonium chloride, PEG-4 dilaurate, lanolin oil, petrolatum, mineral wax, butocouazole nitrate, haloprogin, clotrimazole, O-[(2-hydroxymethyl)methyl]guanine, alclometasone dipropionate, betamethasone valerate, isopropyl myristate MSD, anthralin, methoxsalen, coal tar, 2-(acetyloxy)-9-fluoro-1′,2′,3′,4′-tetrahydro-1-hydroxypregna-1,4-dieno-[16,17-b]naphthalene-3,20-dione, 21-chloro-9-fluoro-1′,2′,3′,4′-tetrahydro-11b-hydroxypregna-1,4-dieno-[16z,17-b]naphthalene-3,20-dione, allantoin, salicylic acid, isopropyl myristate, ascorbic acid, retinol, zinc pyrithione, benzophenone-3, a fragrance, glycolic acid, hyalauronic acid, hydrogen peroxide, a protein, an enzyme, an astringent, a surfactant, tocopherol, butein, hydroquinone, kojic acid, jojoba oil, an alpha or beta hydroxy acid, and mixtures thereof.

21. The composition of claim 1 wherein the polymeric microparticles further comprise a barrier layer in an amount of about 10% to about 70%, by total weight of the loaded microparticles.

22. The composition of claim 21 wherein the barrier layer comprises a compound selected from the group consisting of a poly(acid), a polyol, a salt of a C₈-C₂₀ fatty acid, an alkanolamide, a water-soluble polymer, a biological polymer, a gum, a carbohydrate, a cellulose derivative, a sorbitan derivative, and mixtures thereof.

23. A method of indicating that a personal care, pharmaceutical, wash, oral care, or household cleaning composition has been properly applied comprising:
a) providing a composition having a visual indicator loaded onto polymeric microparticles, and
b) applying the composition a substrate, wherein the microparticles release the visual indicator and generate a color or a color change indicating that the composition has been properly applied.

24. The method of claim 23 wherein the visual indicator comprises a pigment, a dye, an acid-base indicator, a natural food color, a fluorescent compound, or mixtures thereof.

25. The method of claim 23 wherein the substrate is animate.

26. The method of claim 23 wherein the substrate is inanimate.
